(19) United States
(12) Patent Application Publication
(10) Pub. No.: US 2019/0298486 A1
(43) Pub. Date: Oct. 3, 2019

(54) CONNECTION TIP FOR DENTAL ELECTRONIC DEVICE FOR UNIVERSAL APPLICATION

(71) Applicant: APOLLINA, Alixan (FR)

(72) Inventors: Baptiste LAGRANGE, Valence (FR); Pierre DUTHOIT, Malissard (FR)

(21) Appl. No.: 15/992,879

(22) Filed: May 30, 2018

(30) Foreign Application Priority Data
Mar. 30, 2018 (FR) 1852807

(51) Int. Cl. A61C 1/00; A61C 1/07; A61M 5/32; A61M 19/00; A61C 1/14

(52) U.S. Cl. CPC A61C 1/0015; A61C 1/07; A61M 5/32; A61M 2205/6063; A61C 1/14; A61C 2204/005; A61M 2205/6018; A61M 19/00

(57) ABSTRACT

Disclosed is a connection tip for a dental device for universal application, including a hollow cylindrical body including, on the one hand, a fastener for its retention inside the dental device and, on the other hand, a recognition unit which are activated during the connection of the tip inside the device and allow initializing predetermined control unit depending on the profile of the connection tip.

The present invention relates to a range of connection tips for a dental electronic device for universal application with controlled thrust allowing the positioning of different capsules containing, for example, either a biomaterial or an anesthetic.

There are various types of applicators, manual or electric shaped as a pistol, pen or syringe. U.S. Pat. Nos. 4,295,828 and 5,306,147 divulge manual applicators in the shape of a pistol and patents FR2535206 and U.S. Pat. No. 4,693,684 divulge applicators in the shape of a pen. Said applicators are, on the one hand, capable of receiving different heads that can be changed depending on the material to be applied and, on the other hand, consist of a mechanical thrust system whose application force is controlled by the practitioner by pressing more or less strongly on the thrust mechanism.

As regards the electric applicators according to U.S. Pat. Nos. 3,395,704 and 6,159,161, it should be noted that the latter generally consist of a thrust device having only one pushing force allowing to apply, for example, only anesthetic liquids.

It should be noted that there are many applicators that can be classified into 3 major categories namely: those in the shape of a pistol, a syringe or a pen.

It should be noted that the applicators in a shape of a pistol or syringe pose ergonomic problems which result in:
Discomfort for the practitioner and his patient;
Poor quality of treatments due to application defects;
Cabinet clutter (too much to store) with an impact on their good organization and a considerable loss of time.

The use of applicators in the shape of pistols or syringes causes numerous postural imbalances, namely:

The hand is away from the application area;
The dental surgeon has no point of support;
The wrist is not in the axis of the forearm (pinching of the carpal tunnel);
The elbow joint is closed and away from the body, creating an opening of the shoulder;
The head is twisted to properly visualize the area of care. The trapezius is forced to compensate for this movement.

As a consequence, practitioners suffer from a complete panel of musculoskeletal disorders (MSDs):

Cervicalgia;
(Rotator cuff syndrome;
Lateral and medial epicondylitis;
Compression of the ulnar nerve in the epitrochleo-olecranon trough;

Compression of the radial nerve in the Frohse arcade (or radial tunnel);
Tendinitis of the extensors of the hand and fingers;
Tendinitis of the flexors of the hand and fingers;
De Quervain's disease;
Carpal tunnel syndrome;
Compression of the ulnar nerve in Guyon's box;
Raynaud's syndrome from the vibration of the instruments;
Peripheral neuropathies from the vibration of the instruments;
Arthrosis of the elbow, wrist and fingers.

These disorders appear quickly in the career of the dental surgeon since 70% of them complain of pain in the upper limb after only 3 years of practice.

On the other hand, if we examine a complete care sequence for a dental cavity with all the instrumentation known to date, we notice a series of malfunctions that can be attributed to the use of poor tools.

When a practitioner performs this care protocol, we notice the following malfunctions:
The dental surgeon spends more time out of the mouth than on the work area;
Whenever he has to change his instrument, he performs a traumatic rotation of the spine to turn towards the bench behind him, where the instruments he could not put on the plate in front of him are located;
He regularly interrupts his concentration to look in the drawers while the patient closes his mouth, polluting the operative site with saliva;
He must look for up to eight instruments for the care of a single dental cavity;
He has absolutely no tactile feel of the application and therefore poorly controls his gesture.

We also note that the economic stakes of profitability of dental practices have become ubiquitous in the practice of dental surgeons.

They continually seek to optimize protocols to save time in all management phases of the practice. The objective is to optimize the work at the chair, improving synchronization with the assistant, and this in a concern for comfort for everyone.

For this purpose, it is therefore very simple, on the one hand, to consolidate in a cassette all the instruments necessary for an operative act and, on the other hand, to consolidate in a tray all consumables needed for the same act.

The cassette and the tray corresponding to the act are placed on the worktops. All the material is found in these 2 containers, allowing the practitioner and the assistant to avoid looking for instruments in the drawers.

However, the current applicators in the form of a pistol or a syringe cannot be integrated into the cassettes because they are too bulky.

Moreover, the multiple systems clutter the cabinets. This has a negative impact on the operation of the organization, both for the practitioner during a treatment and for his assistant during the cleaning of the instruments.

The dental electronic device for universal application with controlled thrust according to the present invention has been optimized to improve both the instrumental care sequence and the intrinsic quality of the treatment.

Also, the dental electronic device design for universal application with controlled thrust according to the present invention has been adapted so that it fits perfectly into the treatment cycle of instruments, which allows optimizing the overall operation of dental offices and significantly increasing profitability.

Finally, the subject of the present invention also relates to connection tips for a dental device for universal application.

The connection tip for universal application dental device according to the present invention consists of a hollow cylindrical body comprising, on the one hand, fastening means for its retention inside the dental device and, on the other hand, recognition means that are activated when connecting the tip inside said device for initializing predetermined control means depending on the profile of the connection tip.

The connection tip according to the present invention comprises a hollow cylindrical body having an internal bore and, on its outer periphery, locking means which are constituted by a quarter-turn bayonet system whose pins, extending from said cylindrical body, cooperate with complementary locking means of the dental device.

The connection tip according to the present invention comprises a hollow cylindrical body whose internal bore has, on the side of the locking means, an opening for the passage and introduction of the control means of the dental device.

The connection tip according to the present invention comprises recognition means which consist of a code analyzed by optical sensors placed on the electronic card and designed for initializing the program corresponding to the tip locked inside the dental device.

The connection tip according to the present invention comprises recognition means which consist of an imprint on the outer face of the cylindrical body of a Tag or Rfid code which is analyzed by remote sensors.

The connection tip according to the present invention comprises recognition means which consist of pads arranged on the periphery of the tip which activate a switch disposed on the electronic card and designed for initializing the program corresponding to the tip locked inside the elongated body.

The connection tip for performing simple or vibration anesthesia according to the present invention comprises a hollow cylindrical body having, opposite the first opening, a threaded head comprising in its center a small diameter hole opening inside the internal bore, said threaded head used for fastening a needle holder, part of the needle cooperating with the hole and coming inside the bore to tightly cooperate with a capsule filled with anesthetic liquid and provided with a push rod coming into contact with the axis when said tip is locked inside the dental device.

The connection tip for performing simple or vibration anesthesia according to the present invention comprises a hollow cylindrical body having, on its outer periphery, a sheath that can slide between an open position and a closed position to cover the threaded head and protect the needle holder.

The connection tip for injecting composites according to the present invention comprises a hollow cylindrical body having, opposite the first opening, a second opening allowing the positioning and guiding inside the internal bore of a piston secured to a longitudinal rod around which is disposed a spring, said rod passing through, opposite the opening, a double-spout shaped body integral with the hollow cylindrical body and allowing the positioning and retention of a capsule containing a composite material.

The connection tip for injecting composites according to the present invention comprises a hollow cylindrical body having, opposite the first opening, a simple spout shaped body having a single receiving space traversed by the piston rod for the positioning and retention of a double-mixing capsule.

The connection tip for injecting silicone according to the present invention comprises a hollow cylindrical body comprising, opposite the first opening, an extension having the profile of an angled tube, while the second opening is closed, after filling said silicone tip, by means of a plunger plug cooperating with the dental device.

The connection tip for injecting liquid according to the present invention comprises a hollow cylindrical body having, opposite the first opening, an extension configured in a straight tube of small diameter allowing the tight fitting of a disposable head.

The connection tip for injecting liquid according to the present invention comprises a disposable head which consists of a sheath extending through a capillary tube of very small diameter for the diffusion of a liquid.

The tip for the vibration treatment according to the present invention comprises a hollow cylindrical body comprising, opposite the first opening, an extension configured as a spout whose free end has a small sphere allowing the transmission in a specific area of the vibrations from a vibrating device of the dental device.

The tip for injecting two-component composites according to the present invention comprises a hollow cylindrical body comprising, opposite the first opening, a spout having an inverted U-shaped oblong opening into which snaps a double-component capsule comprising two parallel pistons actuated by a piston of said connection tip.

The connection tip for filing inter dental space by vibration according to the present invention comprises a hollow cylindrical body which is secured, opposite the fastening means and the recognition means, to a support head comprising, perpendicularly to the horizontal axis of said cylindrical body, two parallel and opposite branches allowing the positioning and retention of an abrasive belt which is vibrated by the vibrating device of said dental electronic device.

The connection tip for vibration abrading according to the present invention comprises a hollow cylindrical body which is secured, in its extension and opposite the fastening means and the recognition means, to a support head comprising in its center an internal bore allowing the positioning and retention of an abrasive wedge with a tapered profile which is vibrated by the vibrating device of said dental electronic device.

The connection tip for the treatment of gums by vibration according to the present invention comprises a hollow cylindrical body which is secured, in its extension and opposite the fastening means and the recognition means, to a support head comprising in its center an internal bore allowing the positioning and retention of a tip which is vibrated by the vibrating device of said dental electronic device.

The connection tip for the dental devitalization of canals by vibration according to the present invention comprises a hollow cylindrical body which is secured, in its extension and opposite the fastening means and the recognition means, to a support head comprising at its end and following a direction perpendicular to that of the horizontal axis of said cylindrical body, a retaining pin allowing the interlocking of a plastic needle with conical profile which is vibrated by the vibrating device of said dental electronic device.

The connection tip according to the present invention comprises a coding device ensuring the angular pre-positioning of each connection tip when it is placed in the elongated body.

The connection tip according to the present invention comprises a coding device which is formed by a flat surface located on the outer periphery of the hollow cylindrical body.

The connection tip according to the present invention comprises a coding device whose flat surface includes an extension going outside the hollow cylindrical body.

The connection tip according to the present invention comprises a coding device whose flat surface is provided in excess thickness relative to the outer periphery of the hollow cylindrical body.

The connection tip according to the present invention comprises a coding device whose flat surface is hollowed out in the thickness of the hollow cylindrical body.

The connection tip according to the present invention consists of a hollow cylindrical body comprising an internal bore and, on its outer periphery, locking means which consist of an elastically deformable system comprising, on the outer periphery of the cylindrical body, at least two resilient blades which become housed, when the connection tip is fastened, in a window with complementary profile provided in the elongated body.

The connection tip according to the present invention consists of a hollow cylindrical body comprising an internal bore and, on its outer periphery, locking means which consist of a magnet system comprising, at the end of the cylindrical body and on its outer periphery, a magnetically charged metal ring magnetized on a magnetically charged metal ring fixed inside the elongated body.

The following description with reference to the appended drawing and given by way of non-limiting example, will allow to better understand the invention, the characteristics it presents, and the advantages that it is likely to provide:

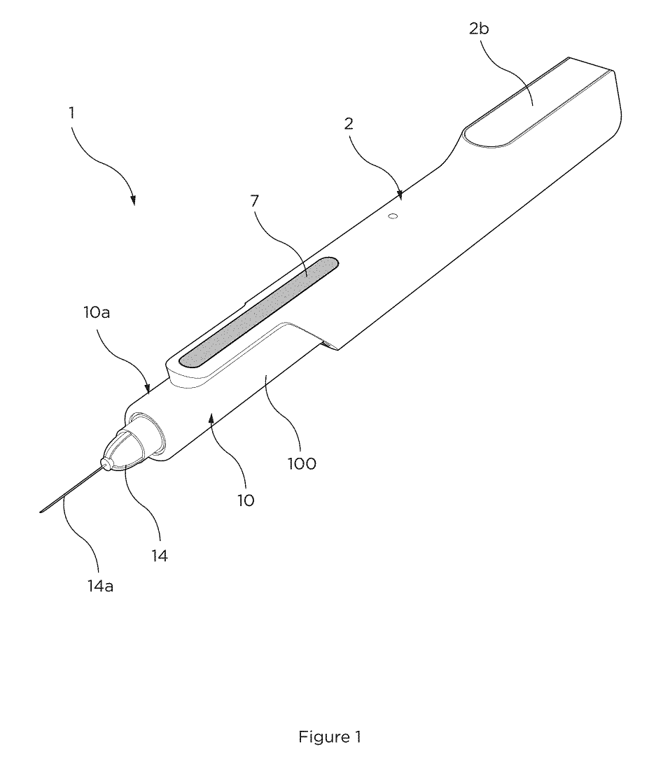

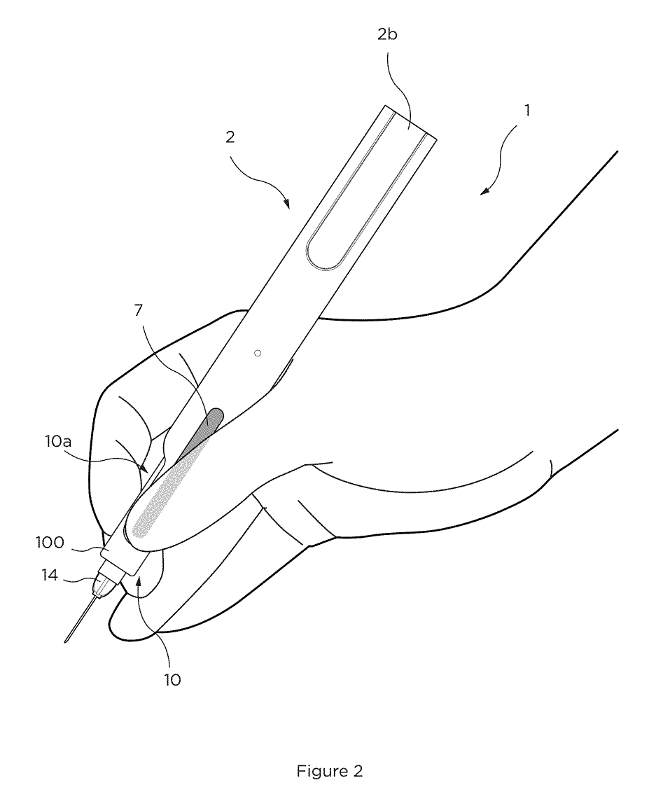

FIGS. 1 and 2 are perspective views showing an example of a connection tip installed in a dental electronic device for universal application according to the present invention.

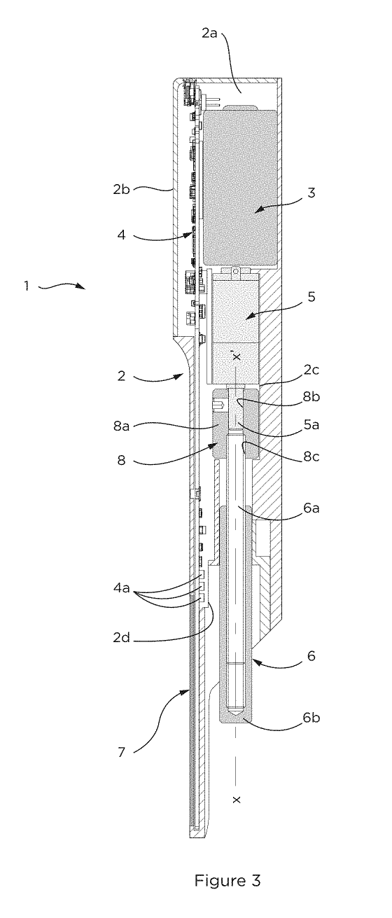

FIGS. 3 to 5 are views showing the connection means for the positioning and securing of the connection tips inside a dental electronic device for universal application according to the present invention.

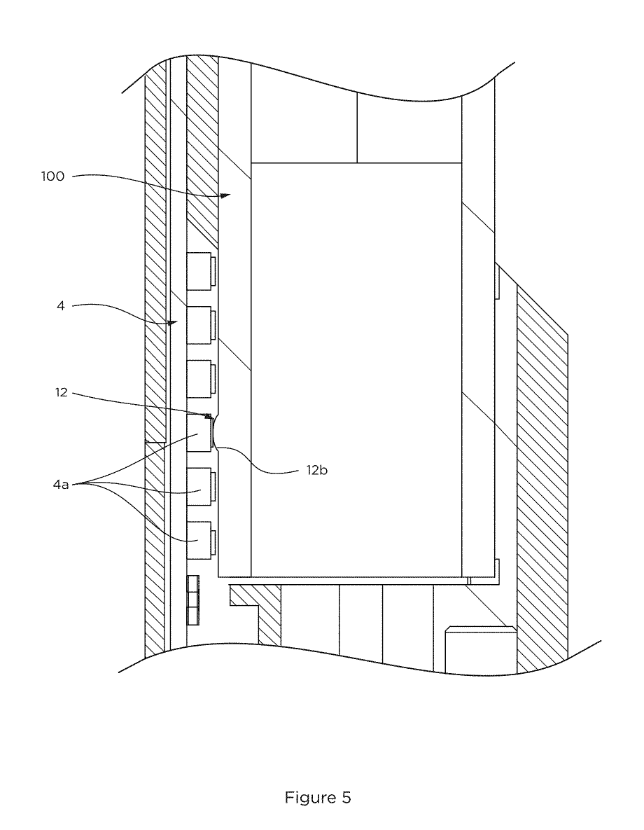

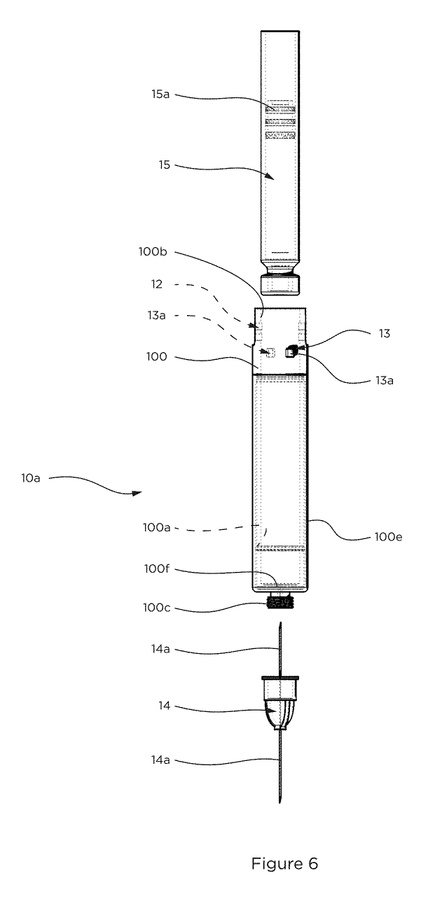

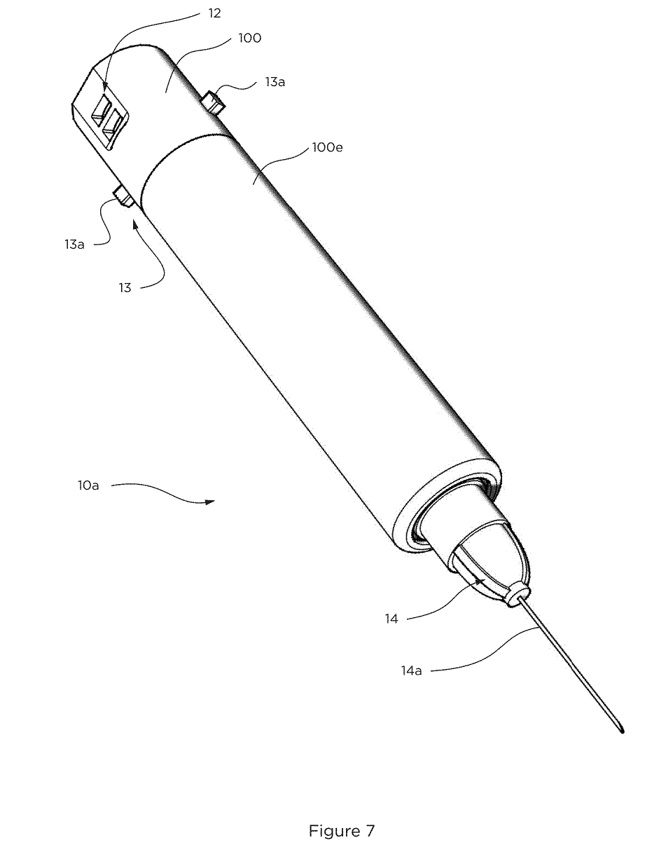

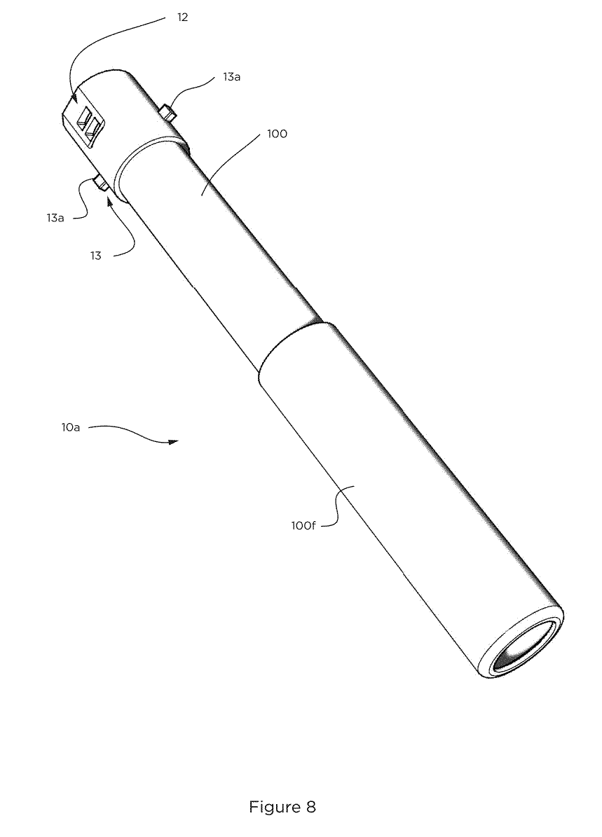

FIGS. 6 to 8 are perspective views showing a connection tip for performing simple or vibration anesthesia according to the present invention.

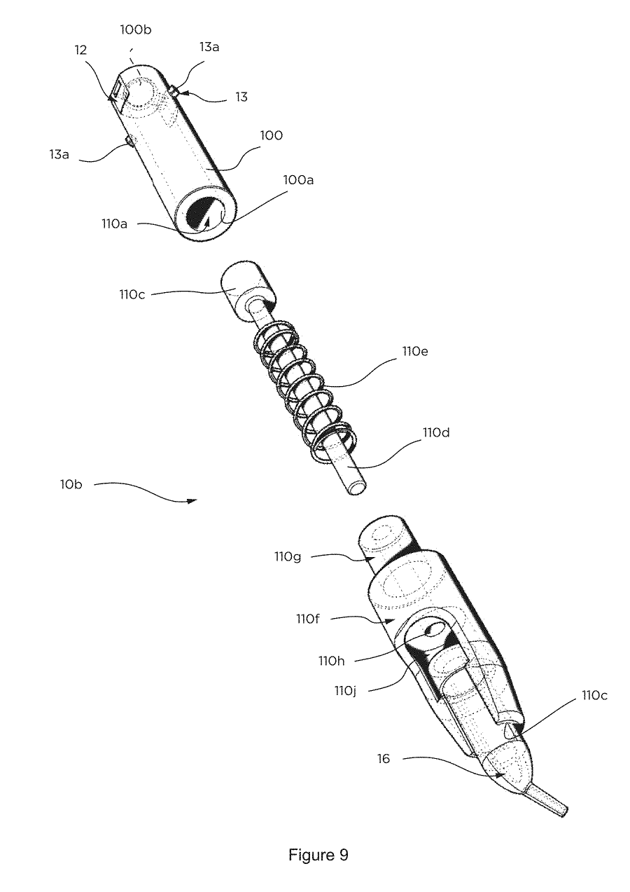

FIGS. 9 to 12 are perspective views showing a connection tip for the injection of composites according to the present invention.

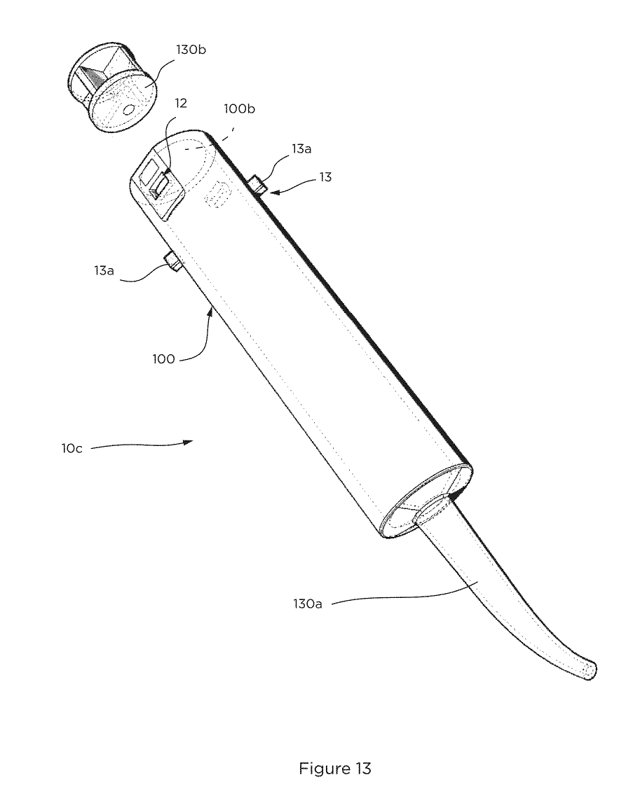

FIG. 13 is a perspective view showing a connection tip for the injection of silicone according to the present invention.

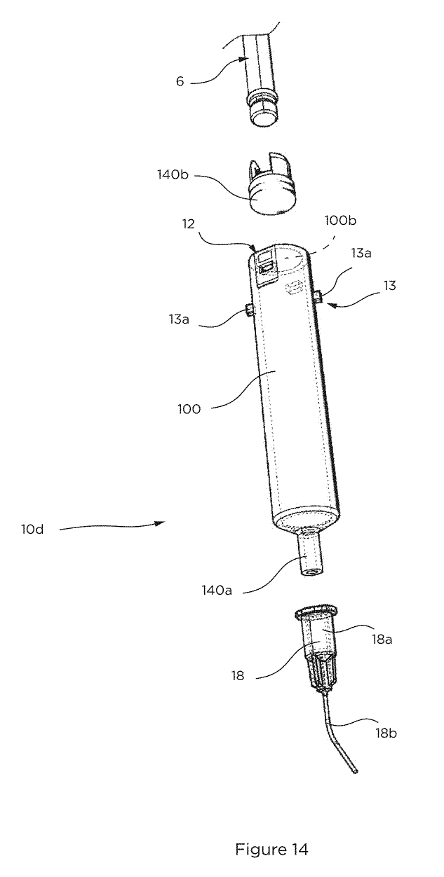

FIG. 14 is an exploded perspective view showing a connection tip for the injection of liquid according to the present invention.

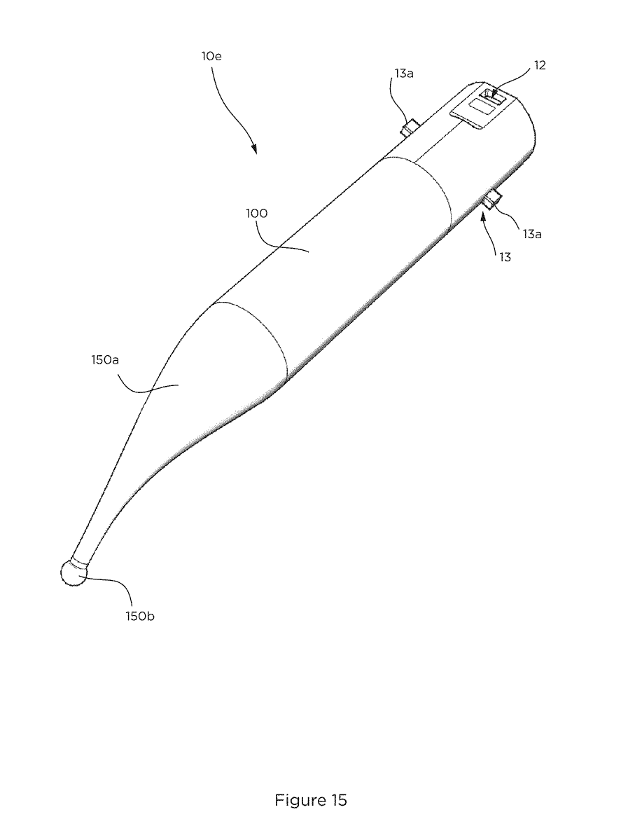

FIG. 15 is a perspective view showing a connection tip for transmitting vibrations according to the present invention.

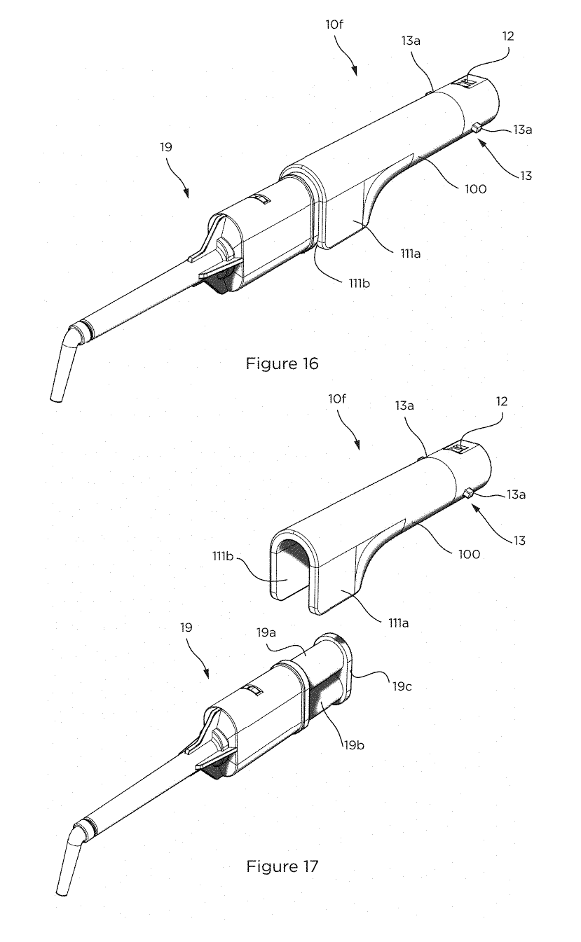

FIGS. 16 and 17 are perspective views showing a variant of the connection tip for the injection of composites according to the present invention.

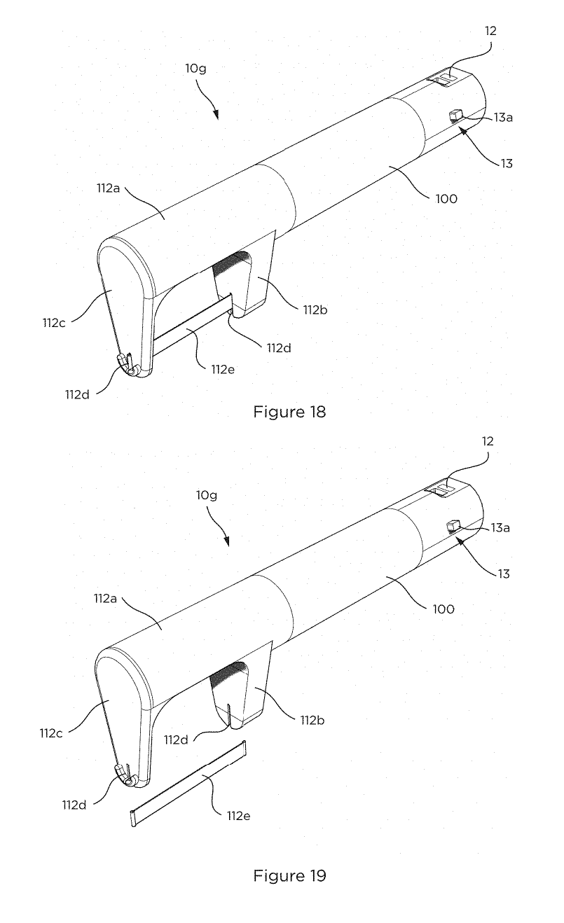

FIGS. 18 and 19 are perspective views showing a connection tip for vibration filing according to the present invention.

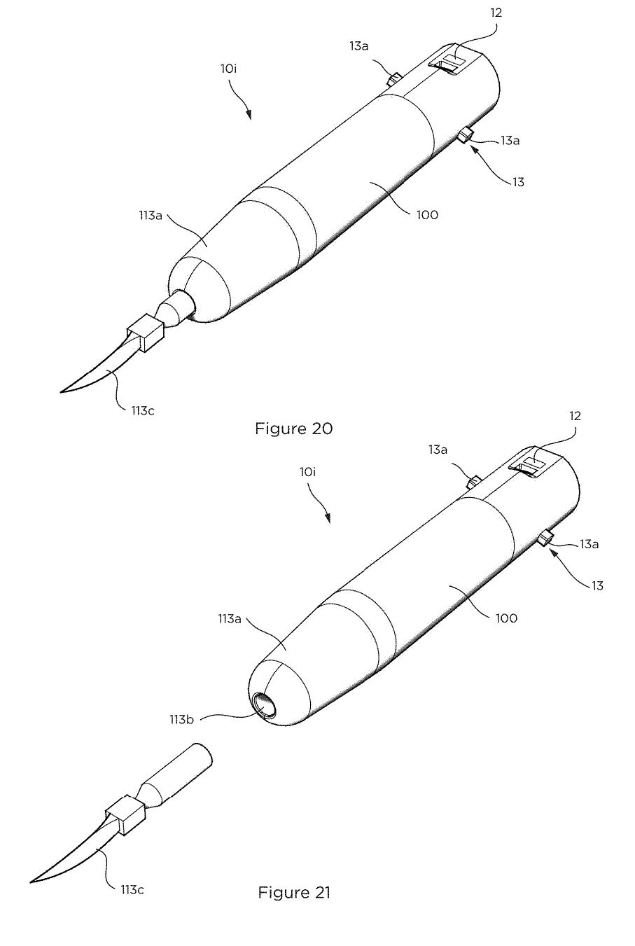

FIGS. 20 and 21 are perspective views showing a connection tip with an abrasive head according to the present invention.

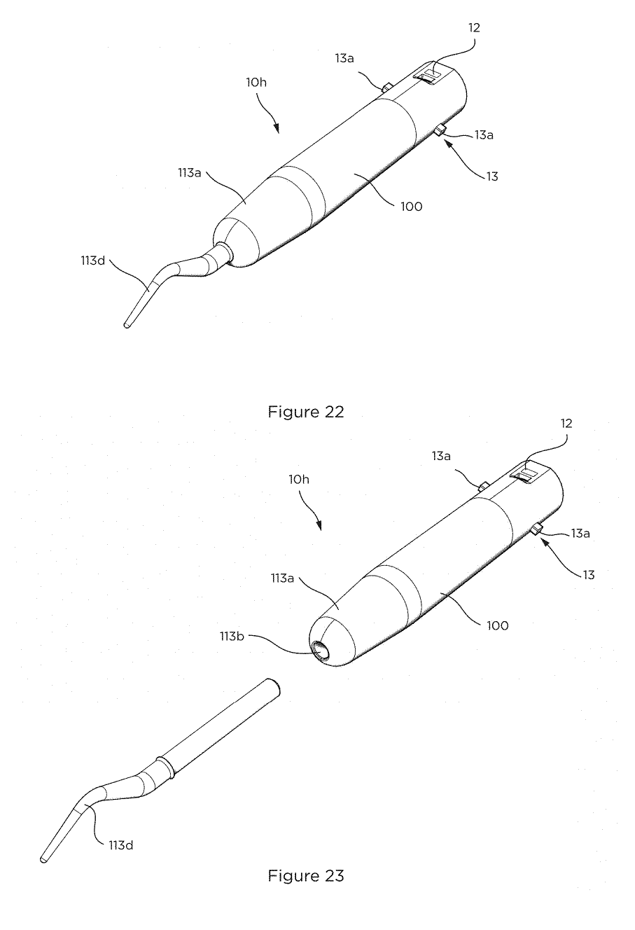

FIGS. 22 and 23 are perspective views showing a variant of the connection tip for transmitting vibrations according to the present invention.

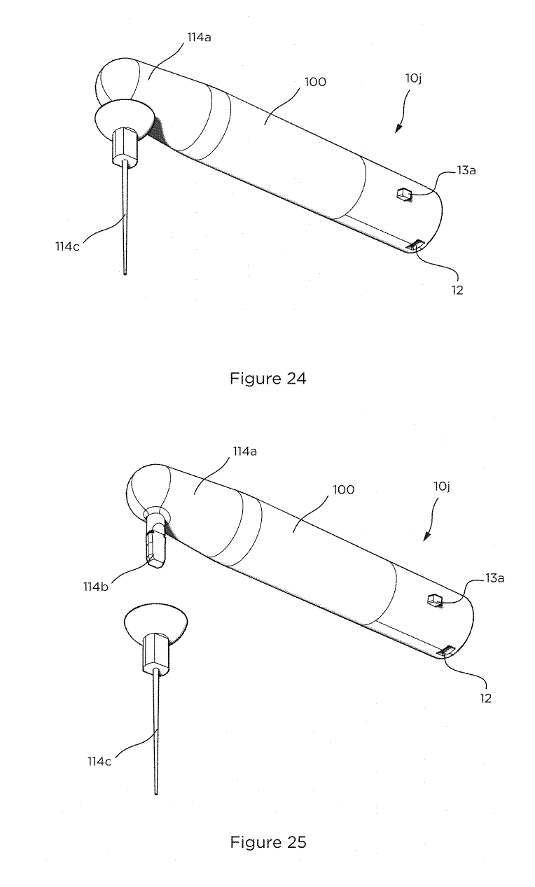

FIGS. 24 and 25 are perspective views showing a connection tip for dental devitalization according to the present invention.

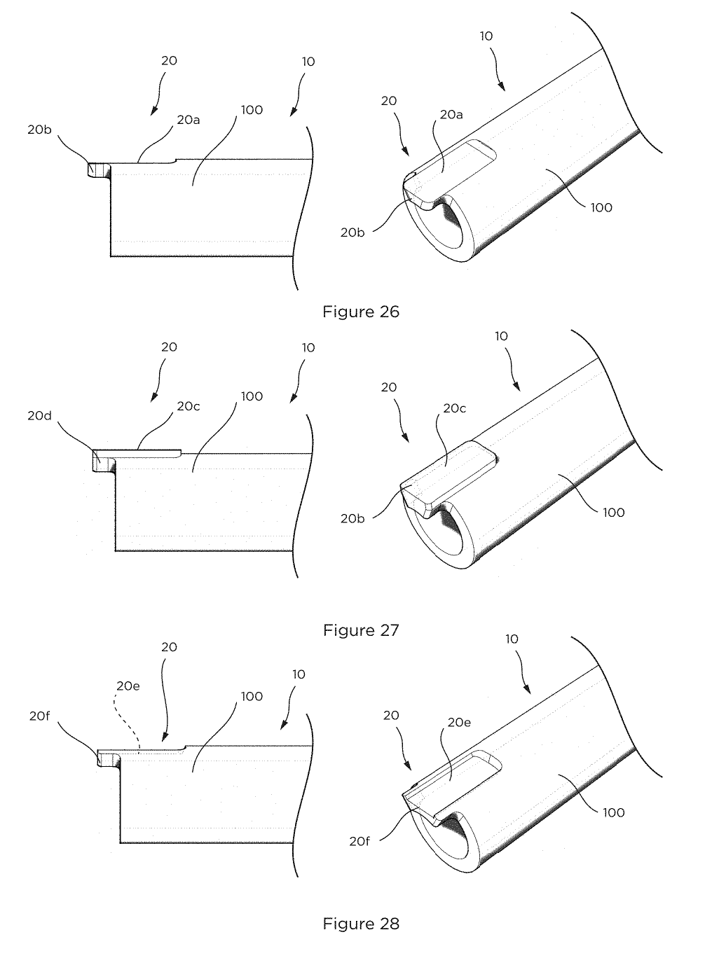

FIGS. 26 to 28 are perspective views illustrating various alternative embodiments of a coding device ensuring the angular prepositioning of each connection tip when it is placed in a dental electronic device for universal application according to the present invention.

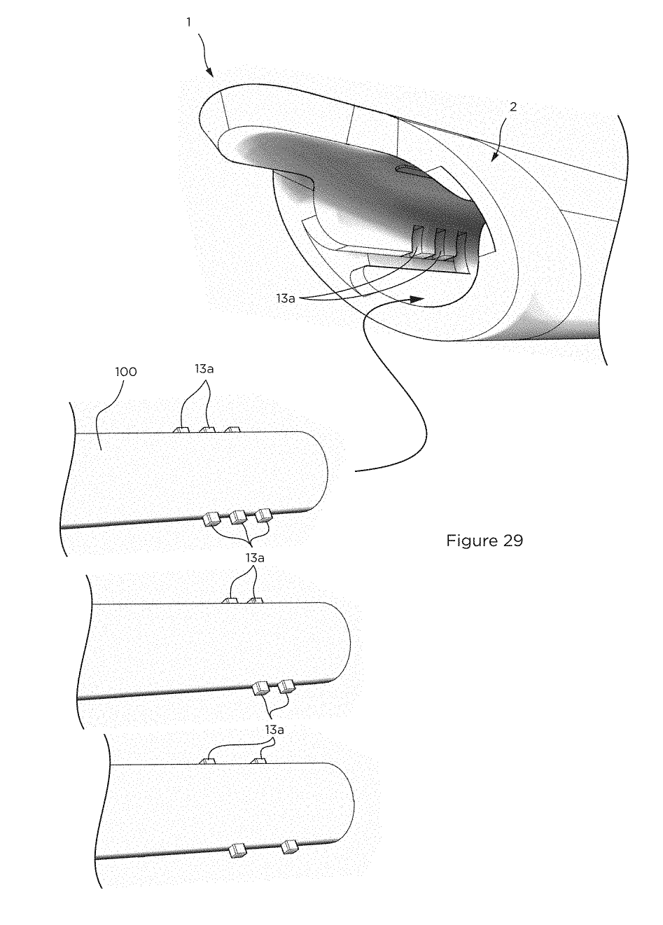

FIGS. 29 to 32 are views showing various alternative embodiments of the locking means of each connection tip within the elongated body of the dental electronic device for universal application according to the present invention.

FIGS. 1 to 3 show a pen-type dental electronic device for universal application with controlled thrust 1 comprising an elongated body 2 carried by a longitudinal axis XX′ and having, on the side of its end furthest from that of the working end, a first space 2a with a substantially cylindrical profile which is closed by a cover 2b.

The first space 2a is provided to receive in its lower part a battery 3 for the power supply of the dental electronic device 1. The battery 3 is positioned inside the space 2a so as to be centered around the axis XX′ of the elongated body.

The upper part of the first space 2a allows the positioning and fastening, above the battery 3, of an electronic control card 4 which is directly powered by said battery.

The elongated body 2 comprises, in the extension of the first space 2a, a guiding base 2c which allows to position, along the longitudinal axis XX′ and in the extension of the battery 3, an electric motor 5 for driving a thrust axis 6 along the longitudinal axis XX′.

The electronic control card 4 has an elongated profile so as to come above the electric motor 5 and the thrust axis 6 to cooperate with a control button 7 positioned in the upper part of the elongated body 2 and, more particularly, at the working end of the dental electronic device 1.

The control button 7 has an elongated shaped profile allowing ease of use for the user depending on the positioning of his hand (FIGS. 2 and 3). It should be noted that the control button 7 consists of an oblong slat 7a of low thickness implanted in the upper part of the body 2 so as to come above the connection zone traversed by the thrust axis 6.

The control electronic card 4 is connected to the electric motor 5 in order to control the latter according to the control program recorded in said card.

The electronic card 4 currently comprises 6 different control programs allowing the user to perform treatments and/or injections of products with different densities in order to perform:
anesthesia;
anesthesia with vibration;
injection of dense composite;
injection of fluid composite;
vibrations alone without injection;
injection of liquid.

The electric motor 5 comprises a drive shaft 5a which cooperates with the thrust axis 6 by means of a connecting element 8 ensuring the transmission of the rotational force of said motor about the axis XX′ to the thrust axis 6 so that the latter can move in translation along said axis XX′.

For this purpose, the connecting element 8 consists of a sheath 8a comprising a first internal bore 8b inside which the drive shaft 5a of the electric motor 5 is immobilized.

The sheath 8a of the connecting element 8 comprises, in the extension of the first bore 8b, a second internal bore 8c which is threaded so as to cooperate with a threaded rod 6a integral, on its outer periphery, with a thermowell 6b constituting the thrust axis 6 of the dental electronic device 1.

The thermowell 6b of the thrust axis 6 is guided in translation inside the elongated body 2 of the dental electronic device 1 so that the latter moves along the axis XX′.

The elongated body 2 comprises, in its internal part, at its working end and above the thrust axis 6, a window 2d that frees access to sensors 4a integral with the electronic control card 4.

The dental electronic device 1 comprises connection means (not shown) for placing it on a charging base ensuring the maintenance and the electric charging of the battery 3.

The dental electronic device 1 comprises a range of connection tips 10 that lock inside said elongated body 2 in the extension of the thrust axis 6.

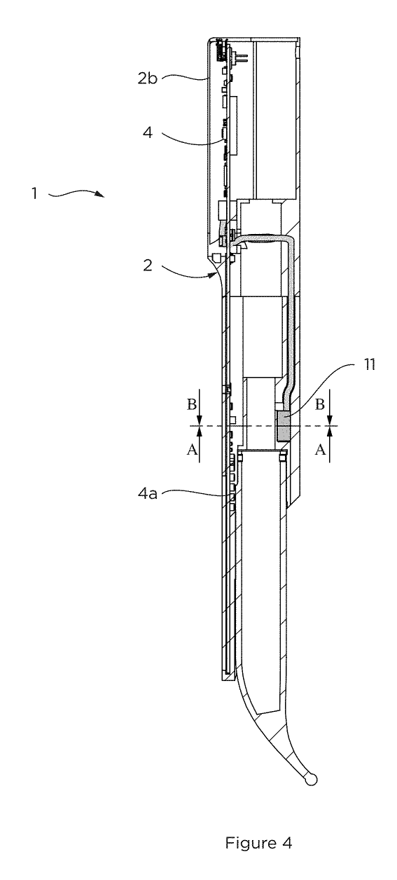

FIG. 4 shows the electronic dental device 1 with controlled thrust comprising, below the thrust axis 6 and near the connection tips 10, a vibrating device 11 connected to the electronic control card 4.

The vibrating device 11 allows the wave diffusion inside the tip in order to put it in vibration to ensure certain treatments or during the diffusion of anesthetic product, for example.

The vibrating device 11 is actuated according to the type of connection tip 10 and the associated control program contained in the electronic card 4.

The range of connection tips 10 comprises currently:
A connection tip 10a for performing anesthesia;
A connection tip 10b for the injection of composites;
A connection tip 10c for the injection of silicone;
A connection tip 10d for the injection of liquid;
A connection tip 10e for vibration treatment;
A connection tip 10f for the injection of dual-component composites;
A connection tip 10g for filing the inter dental space by vibration;
A connection tip 10h for the treatment of gums by vibration;
A connection tip 10i for vibration abrading;
A connection tip 10i for the dental devitalization of canals by vibration.

Each connection tip 10a to 10i consists of a hollow cylindrical body 100 comprising an internal bore 100a and on its outer periphery locking means 13 cooperating with those arranged inside the elongated body 2 located near the thrust axis 6 of the dental device 1 (FIGS. 29 to 32).

The locking means 13 consist, for example, of a quarter-turn bayonet system whose pins 13a extending from the cylindrical body 100 cooperate with housings 13b of complementary shapes formed in the inner wall of the elongated body 2 (FIG. 29).

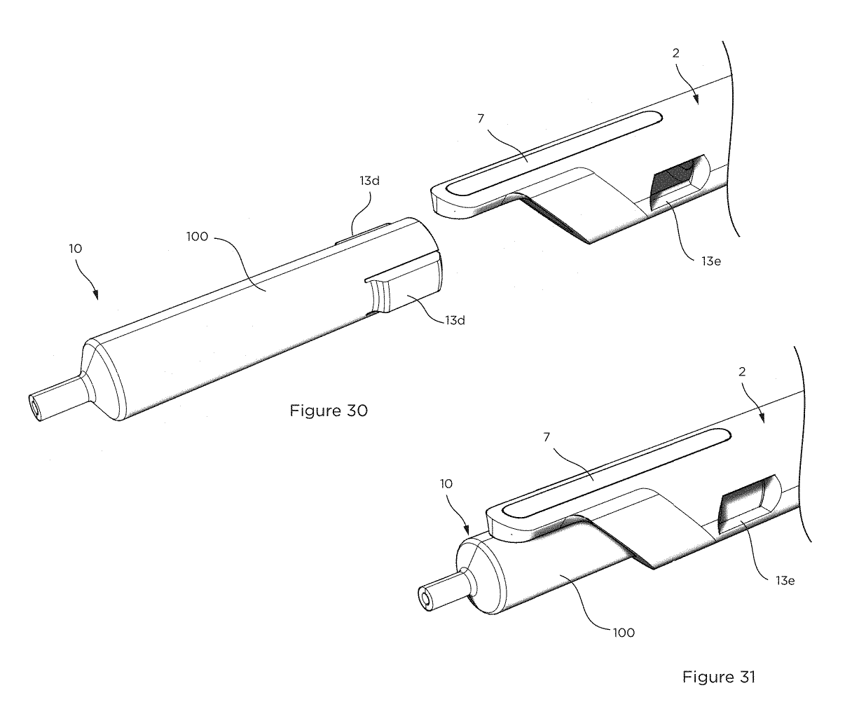

The locking means 13 may be constituted, according to a first embodiment, of an elastically deformable system 13c comprising, on the outer periphery of the cylindrical body 100, at least two resilient blades 13c1 that are housed during the fastening of the connection tip 10a to 10i in a window 13e of complementary profile formed in the elongated body 2 of the dental electronic device 1 (FIGS. 30, 31).

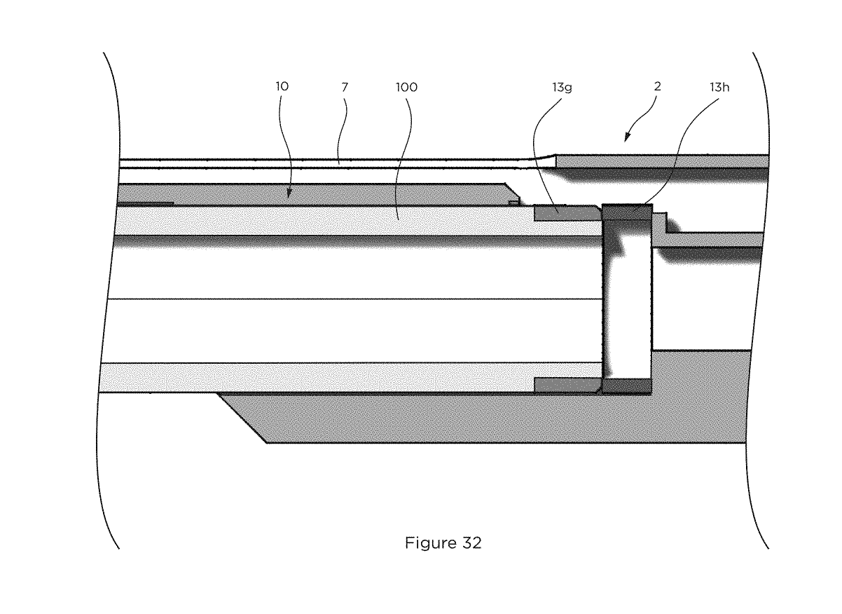

The locking means 13 may be constituted, according to a second embodiment, of a magnet system 13f comprising, at the end of the cylindrical body 100 and on its outer periphery, a magnetically charged metal ring 13g magnetized on a magnetically charged metal ring 13h fixed inside the elongated body 2 of the dental electronic device 1 (FIG. 32).

The internal bore 100a of the hollow cylindrical body 100 comprises, on the side of the locking means 13, an opening 100b allowing passage and positioning into the connection tip 10a to 10i of the thrust axis 6 so as to be able to extract, at the opposite end of said cylindrical body, the product contained in said corresponding tip.

FIG. 5 illustrates the portion of the hollow cylindrical body 100 of each connection tip 10a to 10i comprising recognition means 12 which are activated when the tip is connected inside the elongated body 2 of the dental electronic device 1 to initialize one of the control programs previously integrated in an electronic card 4.

For this purpose, the hollow cylindrical body 100 comprises, on its outer periphery and between the locking means 13, the recognition means 12 which are positioned when locking the connection tip in front of the window 2d arranged in the elongated body 2 so that the latter come into contact with the sensors 4a of the electronic control card 4.

The recognition means 12 may be constituted by a code 12a printed on the outer face of the cylindrical body 100 which is in the locked position of the connection tip 10a to 10j, and identified and read by optical sensors 4a₁ arranged on the electronic control card 4.

The recognition means 12 may be, according to another variant not shown, constituted by printing, on the outer face of the cylindrical body 100, a Tag or Rfid code which is analyzed by remote sensors.

Alternatively, the recognition means 12 may consist of studs 12b positioned in a line or in a star on the outer periphery of the cylindrical body 100, at least one of which coming into contact with the corresponding switch 4a₂ when the connection tip 10a to 10j is in locked position inside the elongated body 2 (FIGS. 6 and 7).

FIGS. 6 to 8 show the connection tip 10a for the realization of simple or vibration anesthesia by means of the dental electronic device 1 according to the present invention.

The hollow cylindrical body 100 of the tip 10a comprises, opposite the first opening 100b, a threaded head 100c having in its center a small diameter hole 100d opening inside the internal bore 100a. The threaded head 100c allows the fastening of a needle holder 14, a part of the needle 14a cooperating with the hole 100d opening inside the bore 100a.

The hollow cylindrical body 100 comprises on its outer periphery a sheath 100e which can slide between an open and a closed position, covering the threaded head 100c in order to protect the needle holder 14.

The cylindrical body 100 comprises, inside the internal bore 100a and in the extension of the threaded head 100c, a cylindrical support 100f traversed by the hole 100d and allowing the tight fastening of a capsule 15 filled with anesthetic liquid and provided with a push rod 15a.

When the capsule 15 is fastened on its support 100f, it also cooperates with the portion 14a of the needle holder 14 previously mounted on the threaded head 100c of the cylindrical body 100.

Before mounting the tip 10a inside the elongated body 2, it is necessary to position on the thrust axis 6 a cylindrical plunger 100h which will be directly in contact with the push rod 15a of the —capsule 15 to allow, during the translational displacement of said thrust axis 6, the ejection of the anesthetic product contained in the latter through the needle holder 14.

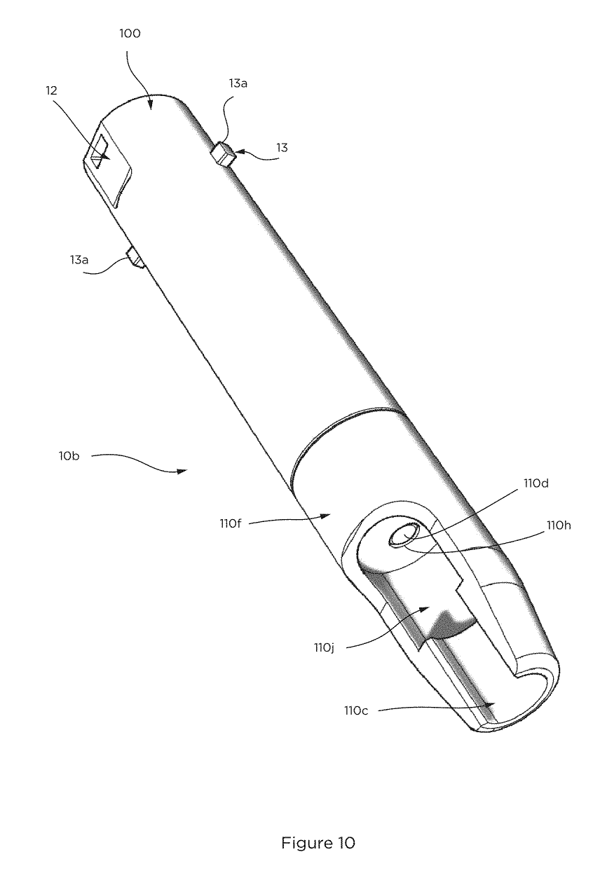

The connection tip 10b, shown in FIGS. 9 and 10, is designed for the injection of composites by means of the dental electronic device 1 according to the present invention.

The hollow cylindrical body 100 of the tip 10b comprises, opposite the first opening 100b, a second opening 110a enabling the positioning into the internal bore 100a of a piston 110c secured to a longitudinal rod 110d around which is placed a spring 110e for maintaining said piston within the cylindrical body 100 in the rest position.

The hollow cylindrical body 100 is secured, opposite the first opening 100b, to a double-spout body 110f having a shoulder 110g with a hole 110h in the center which allows the passage of the rod 110d of the piston 110c.

The double-spout body 110f comprises two receiving spaces 110i and 110i centered on the hole 110h for the positioning and retention of a standard capsule 16 containing a composite material.

When the tip 10b with its cap 16 is fastened inside the elongated body 2 of the dental electronic device 1 and when the thrust axis 6 is actuated by the electric motor 5 driven by the electronic card 4, the latter penetrates inside the hollow cylindrical body 100 through the first opening 110b to come into contact with the piston 110c.

The thrust axis 6 allows the translational displacement of the piston 110c inside the hollow cylindrical body 100 so that the rod 110d of the piston comes into contact with the capsule 16 through the hole 110h formed in the spout-shaped body 110f. The rod 110d exerts a push on the capsule 16 held in the spaces 110i and 110i in order to extract the composite material contained therein.

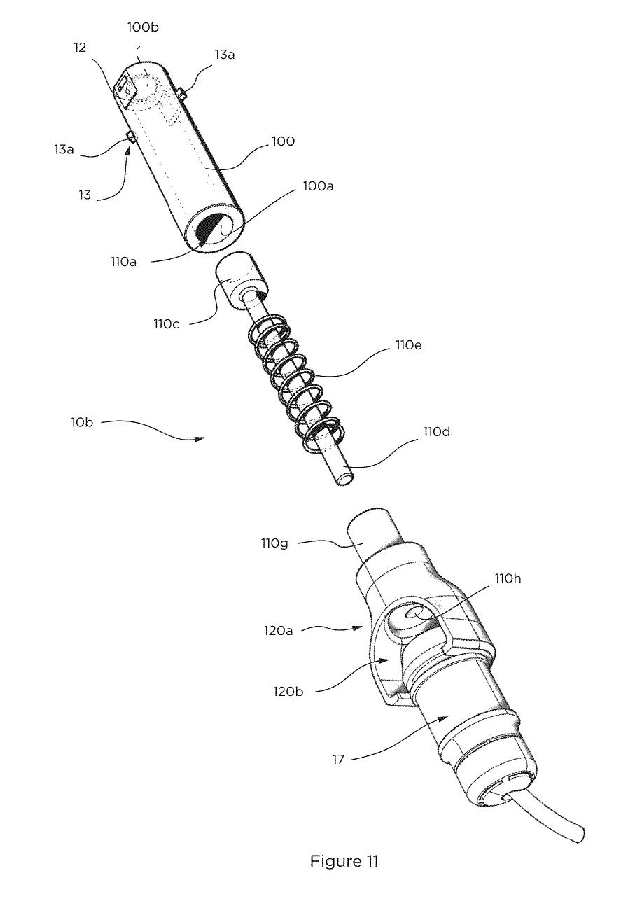

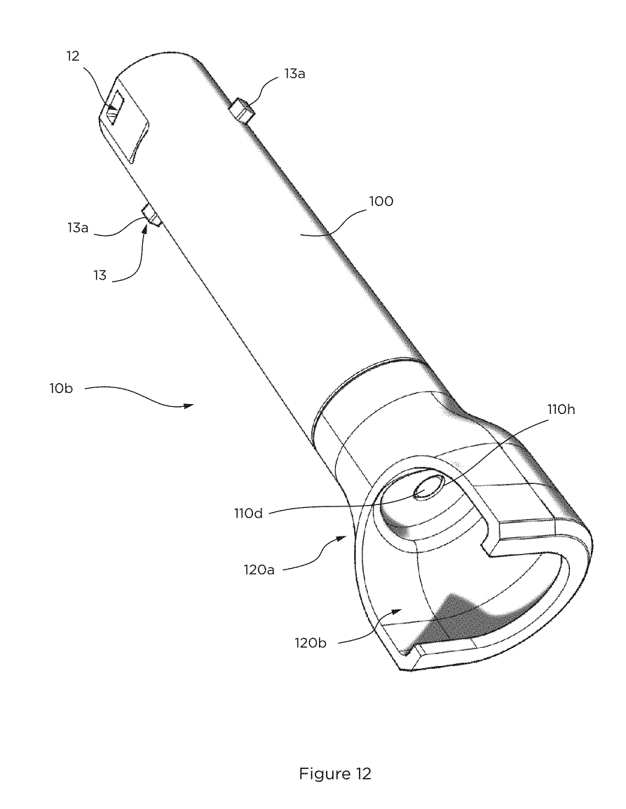

Alternatively, FIGS. 11 and 12 illustrate the connection tip 10b whose hollow cylindrical body 100 is integral with a spout-shaped body 120a having a single receiving space 120b centered on the hole 110h and traversed by the rod 110d of the piston 110c for the positioning and retention of a double-mixing capsule 17.

The connection tip 10c, illustrated in FIG. 13, is designed for silicone injection by means of the dental electronic device 1 according to the present invention.

The hollow cylindrical body 100 of the tip 10c comprises, opposite the opening 100b, an extension affecting the profile of a tube 130a of small diameter with an angled profile.

The hollow cylindrical body 100 is filled through the first opening 110b with silicone or other pasty product. Then, the hollow cylindrical body 100 is closed by means of a plunger plug 130b which is able to move inside the internal bore 100a under the force of the thrust axis 6 when the tip is connected to the elongated body 2.

The thrust axis 6, driven by the electric motor 5 and the electronic card 4, moves the plug 130b inside the cylindrical hollow body 100 for compressing the silicone so that the latter can exit at the free end of the angled tube 130a.

The connection tip 10d, shown in FIG. 14, is designed for the injection of liquid by means of the dental electronic device 1 according to the present invention.

The hollow cylindrical body 100 of the tip 10d comprises, opposite the opening 100b, an extension configured in a straight tube 140a of small diameter allowing the tight fitting of a disposable head 18.

The disposable head 18 consists of a sheath 18a extended by a capillary tube 18b of very small diameter for precise application.

The hollow cylindrical body 100 is filled with liquid through the opening 110b which is then closed by a plunger plug 140b.

The plunger plug 140b comprises fastening means 140c allowing it to snap onto the free end of the thrust axis 6 during fastening of the tip 10d in the elongated body 2 of the dental electronic device 1.

The thrust axis 6, driven by the electric motor 5 and the electronic card 4, moves the plug 140b inside the cylindrical hollow body 100 for compressing the liquid so that the latter can exit at the free end of the straight tube 140a and disposable head 18.

The connection tip 10e, shown in FIG. 15, is designed for transmitting the vibrations of the dental electronic device 1 according to the present invention.

The hollow cylindrical body 100 of the tip 10e comprises, opposite the first opening 100b, an extension configured in a spout 150a whose free end has a small sphere 150b for transmitting the vibrations from the vibrating device 11 of the dental electronic device 1 to a specific area.

FIGS. 16 and 17 show an alternative embodiment for the injection of composites whose connection tip 10f, according to the present invention, comprises a hollow cylindrical body 100 inside which the piston device 110c described above is guided.

The hollow cylindrical body 100 is integral, at one of its ends, with a spout 111a having an inverted U-shaped oblong opening 111b into which a double-component capsule 19 snaps. This capsule 19 is special in that it has two parallel pistons 19a, 19b connected by a plate 19c and in that it imperatively requires operating them at the same time to ensure the mixing of the two components contained within said capsule.

For this purpose, the piston 110c, integral with the rod 110d of the connection tip 10f, is moved in translation by the dental device 1 so that the free end of said rod 110d comes to rest on the plate 19c of the capsule 19 to move the two pistons 19a and 19b at the same time.

The connection tip 10g, shown In FIGS. 18 and 19, is designed for filing by vibration according to the present invention.

The hollow cylindrical body 100 of the connection tip 10g is secured, opposite the fastening means 13 and the recognition means 12, to a support head 112a comprising, perpendicular to the horizontal axis of said cylindrical body, two parallel and opposite branches 112b and 112c.

The parallel and opposite branches 112b and 112c respectively have at their free end fastening means 112d allowing the positioning and retention of an abrasive belt 112e.

When the connection tip 10g is fastened inside the dental electronic device 1, the recognition means 12 enable the operation of the vibrating device 11 of said dental electronic device ensuring the vibration of the abrasive belt 112e.

This connection tip 10g with its abrasive belt 112e allows the dental surgeon to file excess dental dressing or clean inter-dental spaces.

A connection tip 10i with an abrasive head according to the present invention is shown in FIGS. 20 and 21.

The hollow cylindrical body 100 of the connecting tip 10i is secured, in its extension and opposite the fastening means 13 and the recognition means 12, to a support head 113a comprising at its center an internal bore 113b allowing the positioning and retention of an abrasive wedge 113c with tapered profile.

When the connection tip 10i is fastened inside the dental electronic device 1, the recognition means 12 enable the operation of the vibrating device 11 of said dental electronic device, ensuring the vibration of the silicone blade 113c with tapered profile.

This connection tip 10i with its silicone blade 113c with tapered profile allows the dental surgeon to clean the inter-dental spaces by vibration.

A variant of the connection tip 10h, shown in FIGS. 22 and 23, is designed for transmitting vibrations according to the present invention.

The hollow cylindrical body 100 of the connecting tip 10h is secured, in its extension and opposite the fastening means 13 and the recognition means 12, to a support head 113a comprising at its center an internal bore 113b allowing the positioning and retention of a tip 113d.

The tip 113d may have a curved profile to make it easy for the dental surgeon to access the teeth.

When the connection tip 10h is fastened inside the dental electronic device 1, the recognition means 12 enable the operation of the vibrating device 11 of said dental electronic device, ensuring the vibration of the tip 113d.

This connection tip 10h with its tip 113d allows the dental surgeon to bear on the crowns to put them in vibration to improve their positioning.

A connection tip 10i, shown in FIGS. 24 and 25, is designed for the improvement of dental devitalization by vibration according to the present invention.

The hollow cylindrical body 100 of the connection tip 10i is secured, in its extension and opposite the fastening means 13 and the recognition means 12, to a support head 114a comprising a retaining pin 114b at its end and in a direction perpendicular to that of the horizontal axis of said cylindrical body.

The retaining pin 114b allows interlocking, in its extension, a plastic needle 114c with a conical profile.

When the connection tip 10i is fastened inside the dental electronic device 1, the recognition means 12 enable the operation of the vibrating device 11 of said dental electronic device, ensuring the vibration of the plastic needle 114c with a conical profile.

This connection tip 10i with its plastic needle 114c with a conical profile allows the dental surgeon to go inside the canals previously drilled during the devitalization of a tooth in order to transmit a vibration in said canals to remove excess cement.

FIGS. 26 to 28 show various alternative embodiments of a coding device 20 ensuring the angular pre-positioning of each connection tip 10 when it is positioned in the dental electronic device 1.

Each coding device 20 is positioned on the periphery of the hollow cylindrical body 100 of each connection tip 10 and, more particularly, in the same zone as the one carrying the fastening means 13 and the recognition means 12.

The coding device 20 allows angularly pre-positioning each connection tip 10 so that the fastening means 13 and the recognition means 12 respectively cooperate with the complementary elements arranged inside the dental electronic device 1 to allow the fastening and recognition of said connection tip 10.

FIG. 28 represents a first alternative embodiment of the coding device 20 which may consist, for example, of a flat surface 20a located on the outer periphery of the hollow cylindrical body 100.

The flat surface 20a may comprise an extension 20b coming outside the hollow cylindrical body 100.

FIG. 29 shows a second alternative embodiment of the coding device 20 which may consist, for example, of a flat surface 20c added to the external periphery of the hollow cylindrical body 100.

The flat surface 20c may comprise an extension 20d coming outside the hollow cylindrical body 100.

FIG. 30 illustrates a third variant embodiment of the coding device 20 which may consist, for example, of a flat surface 20e hollowed out in the thickness of the hollow cylindrical body 100.

The flat surface 20e may comprise an extension 20f coming outside the hollow cylindrical body 100.

It must also be understood that the foregoing description has been given by way of example only and that it in no way limits the scope of the invention so that, should any of the details of execution described therein be replaced by any other equivalent, this would not be considered as going beyond said scope.

1. Connection tip for dental electronic device for universal application, comprising a hollow cylindrical body (100) that comprises both a fastening means (13) for retention inside the dental device (1), as well as recognition means (12) which are activated when the tip (10) is connected inside said device and which allows to initialize predetermined control means depending on the profile of the connection tip (10a to 10j).

2. The connection tip for a dental device for universal application according to claim 1, wherein the hollow cylindrical body (100) comprises an internal bore (100a) and, on its outer periphery, locking means (13) which consist of a quarter-turn bayonet locking system having pins (13a) extending from said cylindrical body (100) which cooperate with complementary locking means of the dental device (1).

3. The connection tip for a dental device for universal application according to claim 2, wherein the internal bore (100a) of the hollow cylindrical body (100) has, on the side of the locking means (13), an opening (100b) allowing the passage and the introduction of the control means of the dental device (1).

4. The connection tip for a dental device for universal application according to claim 1, wherein the recognition means (12) consist of a code (12a) analyzed by optical sensors (4a₁) placed on the electronic card (4) and allowing to initialize the program corresponding to the tip (10a to 10j) locked in the dental device (1).

5. The connection tip for a dental device for universal application according to claim 1, wherein the recognition means (12) consist of an imprint on the outer face of the cylindrical body (100) of a Tag or Rfid code which is analyzed by remote sensors.

6. The connection tip for a dental device for universal application according to claim 1, wherein the recognition means (12) consist of pads (12b) arranged on the periphery of the tip (10a to 10j) which activate a switch (4a₂) disposed on the electronic card (4) and intended for initializing the program corresponding to the tip (10a to 10j) locked in the elongated body (2).

7. The connection tip for a dental device for universal application according to claim 1, wherein the hollow cylindrical body (100) of the connection tip (10a) for performing simple or vibration anesthesia comprises, opposite the first opening (100b), a threaded head (100c) having in its center a small diameter hole (100d) opening inside the internal bore (100a), said threaded head (100c) allowing the fastening of a needle holder (14), part of the needle (14a) cooperating with the hole (100d) to come out inside the bore (100a) in order to tightly cooperate with a capsule (15) filled with anesthetic liquid and provided with a push rod (15a) coming into contact with the thrust axis (6) when said tip (10a) is locked inside the dental device (1).

8. The connection tip for a dental device for universal application according to claim 7, wherein the hollow cylindrical body (100) of the connection tip (10a) comprises, on its outer periphery, a sheath (100e) that can slide between an open and a closed position to cover the threaded head (100c) and protect the needle holder (14).

9. The connection tip for a dental device for universal application according to claim 1, wherein the hollow cylindrical body (100) of the connection tip (10b) for the injection of composites comprises, opposite the first opening (100b), a second opening (110a) allowing the positioning and guiding inside the internal bore (100a) of a piston (110c) integral with a longitudinal rod (110d) around which is disposed a spring (110e), said rod (110d) traversing opposite the opening (100b) a double-spout body (110f) integral with the hollow cylindrical body (100) and allowing the positioning and retention of a capsule (16) containing a composite material.

10. The connection tip for a dental device for universal application according to claim 9, wherein the hollow cylindrical body (100) of the connection tip (10b) for the injection of composites comprises, opposite the first opening (100b), a single spout-shaped body (120a) having a single receiving space (120b) traversed by the rod (110d) of the piston (110c) for the positioning and retention of a double-mixing capsule (17).

11. The connection tip for a dental device for universal application according to claim 1, wherein the hollow cylindrical body (100) of the connection tip (10c) for the injection of silicone comprises, opposite the first opening (100b), an extension having the profile of an angled tube (130a), while the second opening (110b) is closed, after filling said tip (10c) with silicone, by means of a plunger plug (130b) cooperating with the thrust axis (6) when the tip is connected to the elongated body (2).

12. The connection tip for a dental device for universal application according to claim 1, wherein the hollow cylindrical body (100) of the tip (10d) for the injection of liquid comprises, opposite the first opening (100b), an extension configured in a straight tube (140a) of small diameter allowing the tight fitting of a disposable head (18).

13. The connection tip for a dental device for universal application according to claim 12, wherein the disposable head (18) consists of a sheath (18a) extending by a capillary tube (18b) of very small diameter allowing the diffusion of liquid.

14. The connection tip for a dental device for universal application according to claim 1, wherein the hollow cylindrical body (100) of the tip (10e) for vibration treatment comprises, opposite of the first opening (100b), an extension configured as a spout (150a) whose free end has a small sphere (150b) allowing the transmission in a specific area of the vibrations from the vibrating device (11) of the dental device (1).

15. The connection tip for a dental device for universal application according to claim 1, wherein the hollow cylindrical body (100) of the tip (10f) for the injection of dual-component composites comprises, opposite the first opening (100b), a spout (111a) having an inverted U-shaped oblong opening (111b) into which is snapped a dual-component capsule (19) comprising two parallel pistons (19a, 19b) actuated by a piston (110c) of said connection tip (10f).

16. The connection tip for a dental device for universal application according to claim 1, wherein the hollow cylindrical body (100) of the connection tip (10g) for filing the inter dental space by vibration is secured, opposite the fastening means (13) and the recognition means (12), to a support head (112a) comprising, perpendicularly to the horizontal axis of said cylindrical body, two parallel and opposite branches (112b, 112c) allowing the positioning and retention of an abrasive belt (112e) which is vibrated through the vibrating device (11) of said dental electronic device (1).

17. The connection tip for a dental device for universal application according to claim 1, wherein the hollow cylindrical body (100) of the connection tip (10i) for abrading by vibration is secured, in its extension and opposite the fastening means (13) and the recognition means (12), to a support head (113a) comprising at its center an internal bore (113b) allowing the positioning and retention of an abrasive wedge (113c) with a tapered profile which is vibrated through the vibrating device (11) of said dental electronic device (1).

18. The connection tip for a dental device for universal application according to claim 1, wherein the hollow cylindrical body (100) of the connection tip (10h) for the treatment of gums by vibration is secured, in its extension and opposite the fastening means (13) and the recognition means (12), to a support head (113a) comprising at its center an internal bore (113b) allowing the positioning and retention of a tip (113d) which is vibrated through the vibrating device (11) of said dental electronic device (1).

19. The connection tip for a dental device for universal application according to claim 1, wherein the hollow cylindrical body (100) of the connection tip (10j) for the dental devitalization of canals by vibration is secured, in its extension and opposite the fastening means (13) and the recognition means (12), to a support head (114a) comprising, at its end and in a direction perpendicular to that of the horizontal axis of said cylindrical body, a retaining pin (114b) for interlocking a conical shaped plastic needle (114c) which is vibrated through the vibrating device (11) of said dental electronic device (1).

20. The connection tip for a dental device for universal application according to claim 1, further comprising a coding device (20) ensuring the angular pre-positioning of each connection tip (10a to 10j), when placed in the elongated body (2).

21. The connection tip for a dental device for universal application according to claim 20, wherein the coding device (20) is formed by a flat surface (20a, 20c, 20e) located on the outer periphery of the hollow cylindrical body (100).

22. The connection tip for a dental device for universal application according to claim 20, wherein the flat surface (20a, 20c, 20e) comprises an extension (20b, 20d, 20f) going outside the hollow cylindrical body (100).

23. The connection tip for a dental device for universal application according to claim 20, wherein the flat surface (20c) is provided in an excess thickness relative to the outer periphery of the hollow cylindrical body (100).

24. The connection tip for a dental device for universal application according to claim 20, wherein the flat surface (20e) is hollowed out in the thickness of the hollow cylindrical body (100).

25. The connection tip for a dental device for universal application according to claim 1, wherein each connection tip (10a to 10j) consists of a hollow cylindrical body (100) comprising an internal bore (100a) and, on its outer periphery, locking means (13) which consist of an elastically deformable system (13c) comprising, on the outer periphery of the cylindrical body (100), at least two resilient blades (13d) which are housed, during the fastening of the connection tip (10a to 10j), in a window (13e) of complementary profile provided in the elongated body (2).

26. The connection tip for a dental device for universal application according to claim 1, wherein each connection tip (10a to 10j) consists of a hollow cylindrical body (100) comprising an internal bore (100a) and, on its outer periphery, locking means (13) which consist of a magnet system (13f) comprising, at the end of the cylindrical body (100) and on its outer periphery, a magnetically charged metal ring (13g) magnetized on a magnetically charged metal ring (13h) fixed inside the elongated body (2).
